(19) United States
(12) Patent Application Publication
(10) Pub. No.: US 2005/0273627 A1
(43) Pub. Date: Dec. 8, 2005

(54) Biometrics in issuance of government documents

(76) Inventor: Bruce L. Davis, Lake Oswego, OR (US)

Correspondence Address: DIGIMARC CORPORATION, 9405 SW GEMINI DRIVE, BEAVERTON, OR, 97008, US

(21) Appl. No.: 11/132,709

(22) Filed: May 18, 2005

Related U.S. Application Data
(60) Provisional application No. 60/572,648, filed on May 18, 2004.

(51) Int. Cl. C12P 21/06

(52) U.S. Cl. 713/186

(57) ABSTRACT

A variety of technologies are detailed that can be employed by state departments of motor vehicles (DMVs), and other agencies, e.g., to reduce customer wait time and otherwise improve customer satisfaction, to streamline workflow, and to enhance security.

### RELATED APPLICATION DATA

This application claims priority to provisional application 60/572,648, filed May 18, 2004.

### FIELD

The present disclosure generally relates to methods and systems concerning issuance of identity credentials, such as driver licenses.

### BACKGROUND

The assignee's prior patent filings relating to issuance and use of driver licenses, and related technology, include pending application Ser. No. 10/734,614 (filed Dec. 12, 2003), 60/586,066 (filed Jul. 6, 2004), 60/590,562 (filed Jul. 23, 2004), Ser. No. 10/965,232 (filed Oct. 13, 2004), Ser. Nos. 10/979,770 and 10/980,144 (both filed Nov. 1, 2004); published applications 20050068420, 20050065886, 20050063027, 20050031173, 20040243567, 20040213437, 20040181671, 20040158724, 20040133582, 20040049401, 20040039914, and 20030173406; and issued U.S. Pat. Nos. 5,841,886, 6,389,151, and 6,614,914.

Other background patent documents from third parties include U.S. Pat. No. 5,864,623 (e.g., verifying driver license contents), 20050006460 (e.g., remote personalization and issuance of identity documents), 20040158476 and U.S. Pat. No. 5,888,074 (e.g., driving simulators for training and testing), U.S. Pat. No. 5,717,776 (e.g., improvements to driver license issuance, and on-line renewal), U.S. Pat. No. 4,486,180 (e.g., computer system for administering driver license tests), U.S. Pat. No. 6,681,098 (e.g., computer system for administering tests over the internet), and U.S. Pat. No. 5,915,973 (e.g., remotely proctored exams, including biometric verification).

### SUMMARY

The specification details a variety of technologies that can be employed by state departments of motor vehicles (DMVs), and other agencies, e.g., to reduce customer wait time and otherwise improve customer satisfaction, to streamline workflow, and to enhance security.

### DESCRIPTION OF THE DRAWINGS

FIGS. 1A and 1B show an illustrative process by which driver licenses may be issued.

### DETAILED DESCRIPTION

One aspect of this disclosure addresses widespread customer dissatisfaction with driver license photos. Such a method employs a capture station at which a user can capture plural photo images of him- or herself. (An exemplary capture station is detailed in the assignee's published application 20050068420.) Some or all of these captured images are displayed to the user. The user can then select from the displayed images to indicate which should be used on the ID card. This selection can be made in various ways, such as through a graphical user interface—using a mouse or touchscreen to select among several displayed photos.

Not all of the images captured by the capture station may be technically suitable for use. For example, if biometric information is to be obtained from the selected image (e.g., facial recognition parameters, such as facial eigenvalues), then certain requirements may be imposed on the image (e.g., the image must permit the user's eye pupils to be distinguished). In such instances, images are assessed for technical conformance to requirements prior to presentation to the user for selection. Alternatively, the assessment can be performed after user selection; if the selected image is found to be unsuitable, then the user can be prompted to make another choice.

Biometric information gleaned from the image can be stored in a database record associated with the user and/or user ID, and used later. For example, such information can be used to deter the issuance of multiple drivers licenses to the same individual. When a person applies for a drivers license, their facial parameters can be compared against those in the universe of previous drivers license facial images, to determine whether a license has earlier been issued to someone who looks like the present applicant. If so, suitable further checking can be undertaken. (Such checking for duplicate driver license images is further detailed in various of the assignee's prior patent filings, including certain of the patent applications cited above.)

Alternatively, the biometric information gleaned from the image can be used in conjunction with data stored on the card. For example, the biometric facial image or various characterizations of it can be stored in a watermark, bar code, magnetic stripe, or other data storage feature on the card. (Such biometric characterizations of faces—as well as other human features—are well understood by artisans in the field. Examples include characterizing a face by locations of key points, by principal components analysis, by eigenvectors, by local feature analysis, by elastic bunch graph methods, by eigenface techniques, etc.) Or a hash can be formed from such parameters (perhaps in conjunction with other data) and similarly stored on the card. Or the hash can be used as a cryptographic key that is applied to other data stored on the card. Etc., etc.

In some embodiments, the image selected by the user can be made available for purposes other than for use on the photo ID card. Examples including use as a passport photo, emailing by the user to friends, creation of novelty items bearing the image, incorporation into greeting cards, and use on a corporate photo ID badge.

In the example given above, biometric information is derived from the image. In other arrangements, biometric information can be obtained otherwise. For example, it can be obtained from the user (e.g., sensing a user fingerprint). Or it can be obtained from another trusted source, such as machine readable data conveyed by a different identity credential (e.g., the TSA's Transportation Worker Identity Credential, aka a TWIC card, or the Department of Defense's Common Access Card, aka CAC). Such non-image-derived biometric information can be processed and stored on the card, or otherwise used in connection with data stored on the card, or may be stored remotely in a database in association with the card or the user.

Before the identification card is issued to the applicant, the applicant's identity may be corroborated by reference to biometric information. Thus, for example, if the applicant presents a passport as evidence of identity, and the passport includes fingerprint data, the fingerprint of the applicant can be checked with the data in the passport as part of verifying identity.

Biometric information can also be used in conjunction with the knowledge test that is required when applying for certain credentials. For example, to secure a driver license, a test of traffic and safety rules is commonly required.

Such tests are frequently administered by computer, with questions (commonly multiple choice questions) presented on a screen, to which the applicant responds by typing, pointing, or clicking. However, other testing can also be used (e.g., presenting virtual reality event simulations of driving in a simulator environment, and judging the applicant's responses and other behavior during the simulation.)

The computer system used to administer the test can also be equipped with the capability to biometrically note the identity of the test-taker. This can be done in various ways.

For example, biometric information earlier-obtained in connection with the application process (e.g., facial parameters from photograph, retinal or corneal scan data, fingerprint information, voiceprint information, biometrics from other trusted sources, etc.) is re-sampled at the testing station, and the two data sets are compared. If they do not match, an error condition is noted. (This may entail flagging suspected fraud to a proctor of the test—if present, and refusing to administer the test to that applicant.)

The re-sampling of biometric information can be repeated during and at the end of the testing, and sampling results compared. If any discrepancy is noted with the earlier-collected information, an error procedure is again followed.

In some cases biometric information sampled from the applicant during testing is compared with other information sometime after the test is completed. (This may be the case, e.g., if testing occurs early in the credential issuance process—before any other acquisition of applicant biometrics.) Again, if the biometrics acquired at the time of testing do not correspond to those obtained otherwise in the process, issuance of the credential can be denied.

In still other arrangements there may be no biometric comparison. Instead, biometrics sampled during testing can be used in connection with issuance of the credential. For example, at the beginning (and/or end, and/or intermediate point) of the testing, the computer can instruct the applicant to place their thumb or other finger(s) on a fingerprint sensor. The fingerprint data obtained in this process is the biometric data that is then stored, or used, in conjunction with issuance of the credential. (If the testing procedure involves sampling the fingerprint twice or more, the different sets of fingerprint data can be compared to ensure that they match. If not, an error condition can again be noted, and appropriate steps followed.) A similar biometric validation can be done relatively unobtrusively via a camera mounted on the test workstation or somewhere else in proximity to the test subject.

Customer satisfaction may be further increased by enhancing the speed and accuracy of the application/issuance process. One opportunity for doing so is by allowing the applicant to enter certain information (e.g., full name, residence address, birth date, hair color, eye color, organ donor preferences, etc.) on a computer terminal that is linked to the issuance computer system. When the applicant is thereafter interviewed by an agency employee, this information is already entered into the issuer's computer system—likely with fewer typographical errors than would be the case if the employee, who not as familiar with such information, entered the data.

In one arrangement, such data entry would be performed by the applicant using a keyboard on a terminal in the agency office, or a voice recognition system. However, other arrangements can also be used.

For example, the applicant may complete this data-entry part of the process from a site remote from the government agency office (e.g., home). Using a web browser, the applicant may load a web page from the issuing agency (e.g., DMV office) which solicits the personal information (e.g., by a series of fill-in-the-blank questions), and transmits the entered data (e.g., by a secure sockets layer/HTTPS connection) to the agency.

In such arrangements, the agency web server (or other computer to which the information is directed) can note from the packets of received applicant data the IP address of the originating applicant computer, and optionally the addresses of intervening computer servers through which the data passed. This IP address information can be used as part of the applicant verification procedure. Thus, for example, if personal information from an applicant purporting to reside in Portland, Oreg. appears to originate from a computer in Austin, Tex., it can be flagged as potentially suspect. The agency may refuse to accept such information, or it may subject the applicant to further scrutiny when verifying the applicant's identity (e.g., requiring further proof, or conducting further verification operations).

The personal information entered by an applicant from the remote location typically includes the applicant's residence telephone number. To aid in applicant verification, the agency may require that the applicant telephone an automated agency telephone service within a set period (e.g., five minutes) of submitting the personal information over the web. (This instruction can be presented to the applicant on a web page displayed during the web-based data collection process.) When connected, the applicant can be prompted to enter an identifier—such as a user-defined password, social security number or telephone number—by which the phone call may be correlated to the just-submitted personal information. The automated agency telephone service is equipped with caller ID capability, and thereby determines the telephone number that originated the call. If the originating telephone number does not match the residence telephone number earlier entered for the identified applicant, the agency may again flag the process as suspect, e.g., proceeding as outlined above.

Conventionally, motor vehicle registration certificates have been documents measuring about 5″×8″ that are kept in the glove box of the motor vehicle. (Such certificates are commonly issued by state agencies to memorialize the owner of record of the vehicle, and/or to confirm that any applicable vehicle tax has been paid for the current period.)

According to another aspect, motor vehicle registration certificates are instead printed on a substrate sized for fitting with credit cards in a person's wallet or purse. By this arrangement, if the vehicle is stolen, the vehicle likely will not also include the associated motor vehicle registration document.

In households where there are several drivers for a vehicle, several such documents may be issued—one for each driver.

Title documents for motor vehicles may also be issued in this form.

In a related arrangement, a two-part motor vehicle registration credential is issued. One part is adapted for storage in the vehicle, and may be of the familiar half-sheet format (e.g., 5″×8″). The second part is printed on a substrate sized for fitting with credit cards in a person's wallet or purse (e.g., a wallet card). The two parts include first and second data, respectively, that logically link the two portions together. Proof of proper vehicle registration may thus require presentation of both portions, and checking that the first and second data correspond in the expected manner.

In one of many possible embodiments, the first data is a cryptographic key, and the second data is personal information that has been encrypted by this key. Thus, the in-vehicle document can include a machine-readable first data comprising a 128 bit cryptographic key. The wallet card can include machine-readable second data comprising the name of the driver bearing the card, encrypted by that 128 bit key. To confirm correspondence, the key is read from the first document and used to decrypt the second data from the wallet card. The result should yield the driver name printed on the wallet card. If not, then something is amiss and further action may be taken.

The 128 bit key can take any number of forms. For example, it can be an ASCII representation of text data printed on the first document (e.g., vehicle identification number, or vehicle owner name) that has been hashed to yield a 128 bit string.

The machine readable first and second data can be encoded on the first and second documents in various ways, including 1D and 2D barcodes or other visible symbologies, magnetic stripes, digital watermarking, RFID, semiconductor memory, optical memory, fiber fingerprinting, etc.

In a household with several drivers there can be several different wallet cards, one for each driver. Any family member can thereby establish proper registration of the vehicle, and/or their own authorization to drive same.

As is readily apparent to those skilled in the art, myriad other arrangements for logically linking documents (e.g., involving public and private key cryptography, digital signatures, hashes, etc., based on any information printed or encoded on either document) can be devised to meet the requirements of particular applications. Thus, for example, it is straightforward to provide a household having three drivers and four cars with four wallet cards and three in-vehicle documents, which allow each driver to evidence registration of all vehicles, and their authorization to drive same.

Logical linking of an in-vehicle registration document with a wallet card is not the only such possibility. Either document, or both, can also be linked to other documents, such as driver licenses, social security cards, credit cards, debit cards, paper checks, etc.

In one alternative, a driver license is substituted for the wallet card. Machine readable information already present on the license (e.g., magnetic stripe, bar code, or watermark data) is used as the basis for the link. For example, such information can be replicated in the machine readable information on the vehicle document. Or such information can be processed and used—either directly on the vehicle document, or as a decryption key for information (e.g., owner name, VIN) encoded on the vehicle document, etc.

While the arrangement just-described included two cooperating physical documents that are dedicated to evidencing vehicle registration, etc., in other embodiments, a more general purpose document or apparatus may be substituted for either. For example, this functionality can be met by any data carrier device kept in the custody of the person—a device that might also serve as a bank card, credit card, repository of medical history, etc. Such data carrier can comprise a semiconductor memory, a PalmPilot or other PDA, a cell phone, a digital audio/video player, a chip card, etc.

The assignee's patent U.S. Pat. No. 6,882,738 details how physical objects, including vehicle parts, can be encoded with machine readable information—such a Vehicle Identification Number. Thus, the logical linking contemplated herein can extend between a data carrier on a document, and machine readable data etched, e.g., in the glass, of a vehicle itself.

The logical linking of first and second data to authenticate a data carrier, or to establish a relationship between two data carriers, is further detailed in various of the assignee's prior applications—including certain of those referenced above (e.g., 20040049401 and U.S. Pat. No. 6,389,151).

In connection with driver licenses, it should be noted that this credential need not always take the form of a specialized card bearing government-specified indicia and data. It may comprise data stored in a more general purpose data carrier—as just noted. Moreover, the traditional form of a card bearing government-specified indicia and data can be enhanced by the provision of additional indicia or data specified by the user to enhance the aesthetic value to the user and the security of the document. Thus, for example, in addition to a photo of the licensee a driver license may include a photo of the licensee's pet, or car, or spouse.

Such customization data can be provided by the applicant to the issuing authority when applying for a license. In one case, the applicant can download such customization data, e.g., into a department of motor vehicle's computer, from a memory device in the possession of the applicant. Or the applicant can provide the issuing authority with an electronic address (e.g., a URL or IP address) at which the desired customization data is stored. The issuing authority can then integrate the customization data onto the card at the time of its fabrication. For example, a photo of the licensee's spouse can be printed on the card in an available location on either the front or back of the card. Such aesthetic enhancements have been permitted in bank cards and other membership cards.

In driver licenses featuring such customization, the customized feature (e.g., photo) can serve an official purpose. For example, it can be used by the issuing authority as a covert carrier of data. Such data can comprise, e.g., the license number, the licensee name, address, birthday, hair or eye color, state of issuance, restrictions, organ donor information, etc., or a combination of any of the foregoing, or a hash of any of the foregoing. Digital watermarking (steganography), as detailed in applications and patents incorporated by reference, can be used for such covert data encoding, and can later be employed for verifying the document or the identity of the licensee.

The uniqueness of such a card enhances security, and its customization makes it more desirable for consumers. The use of the customer-provided indicia as a carrier of official information makes it harder to counterfeit.

References were made in the foregoing detailed description to licenses being issued by government agencies, such as state departments of motor vehicles. Conventionally, the personnel issuing such licenses have been government employees. However, this need not be the case. For example, a state may contract-out much or all of the driver license issuance process to private contractors, who may charge a fixed price for enumerated services (e.g., providing knowledge tests, issuing learner's permits, issuing driver licenses, and/or renewing driver licenses, in a mall location, with specified hours and specified staffing). Or the contractor may bill for its services on a per-license (or other transaction) basis. In either event, the difference between the private contractor cost and the licensee fees received, would be profit to the state treasury or a state agency. (The data collected during the process would typically belong to the state, and the contractor would be contractually bound to specified privacy terms.) Such arrangements provide savings in state payroll, while maintaining revenue from license issuance.

Some functions may be reserved to state employees, such as the road test for new drivers. Alternatively, this function, too, can be out-sourced.

Such a private contractor could achieve efficiencies by using common resources to serve several different issuing agencies. This is already done in “central issue” printing of licenses, in which a privately-owned facility may print licenses for several different jurisdictions. In another example, it is already common for certain state agencies to host web sites through which their licensees can effect transactions including driver license renewal, driver license change of address, requesting a replacement driver license, requesting custom vehicle plates, providing a practice test in preparation for a driver license knowledge test, motor vehicle registration renewal, and motor vehicle registration change of address. Such web services could be standardized by a private vendor, and offered through a web portal to which licensees of many states could turn for the provision of such services. In some cases, the same web interface may be used by licensees in two or more states. In others, the states may wish to have customized interfaces. In such cases, a common web portal would first ask the user to identify their state. Based on the user response to this query, further common screen displays are customized to correspond to that state, or the user can be linked to customized web pages used exclusively for that particular state.

Similarly, economies can be achieved in providing training materials to new drivers. Most issuing agencies provide both printed and PDF versions of driver license manuals to interested parties. These manuals can be edited and published by a single vendor to serve plural states—providing economies in editing and printing that, again, contribute to state treasuries.

Web services can also increase coordination between different issuing authorities. Consider a driver who is licensed in a first state, and moves to a second state. A license transfer “wizard” web site can present to such an applicant a page through which the applicant enters information (e.g., name, social security number, birth date, current address, former address, former driver license state and number, etc.). The web site, or another computer to which this collected information is passed, can verify at least certain of the entered information. When sufficiently verified (and when other requisites, such as knowledge test and image/biometric collection are completed), the system sends data to the first state regarding cancellation of the former license, and authorizes the issuance of a new license in the second state. All of this may be done remote from any DMV office, e.g., from the applicant's new home.

The verification can include checking at least some of the entered applicant information against information earlier stored in a computer record by the first state. Likewise, in response to notification by this service, the DMV image database in the first state can send a copy of the applicant's image to an image database maintained by the second state. (This image may or may not be used on the new license. But its custody by the second state enhances its ability to check for fraud.)

Likewise, the same system could be used to transfer a vehicle registration from one state to another (or from a buyer to a seller).

Another web service “wizard” is one dedicated to new drivers. Such a web site can provide information about office hours and locations, training manuals, and sample tests (optionally charging a fee). The user enters personal information (e.g., their state, their age, the type license desired, etc.), and the system responds with the information specifically needed by that person.

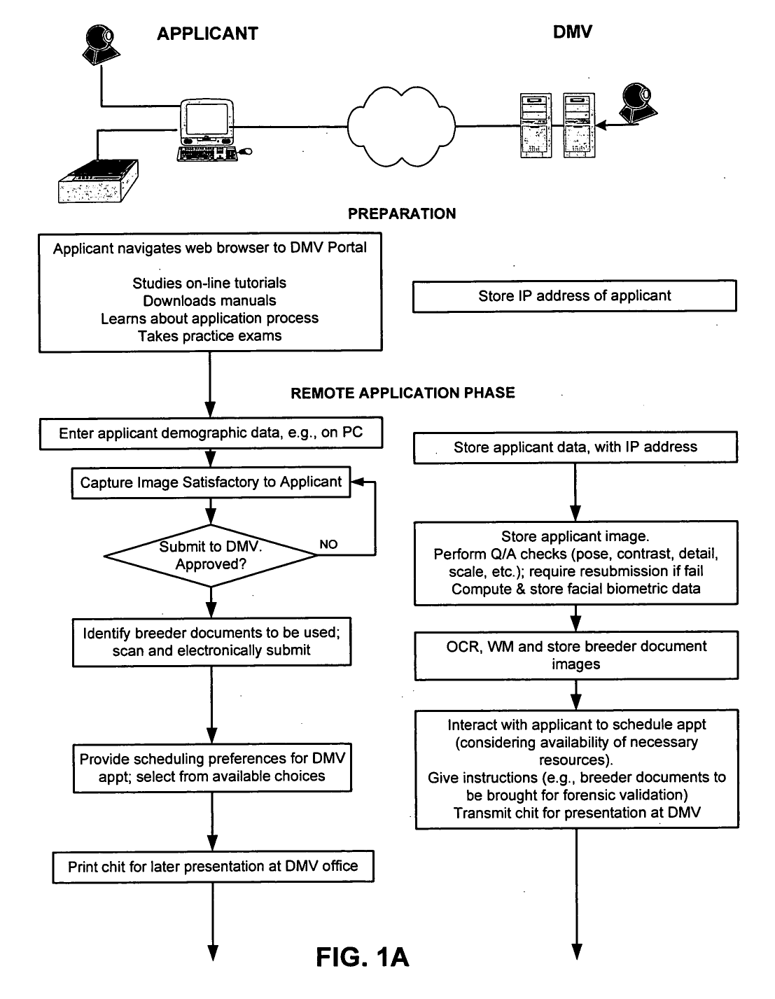

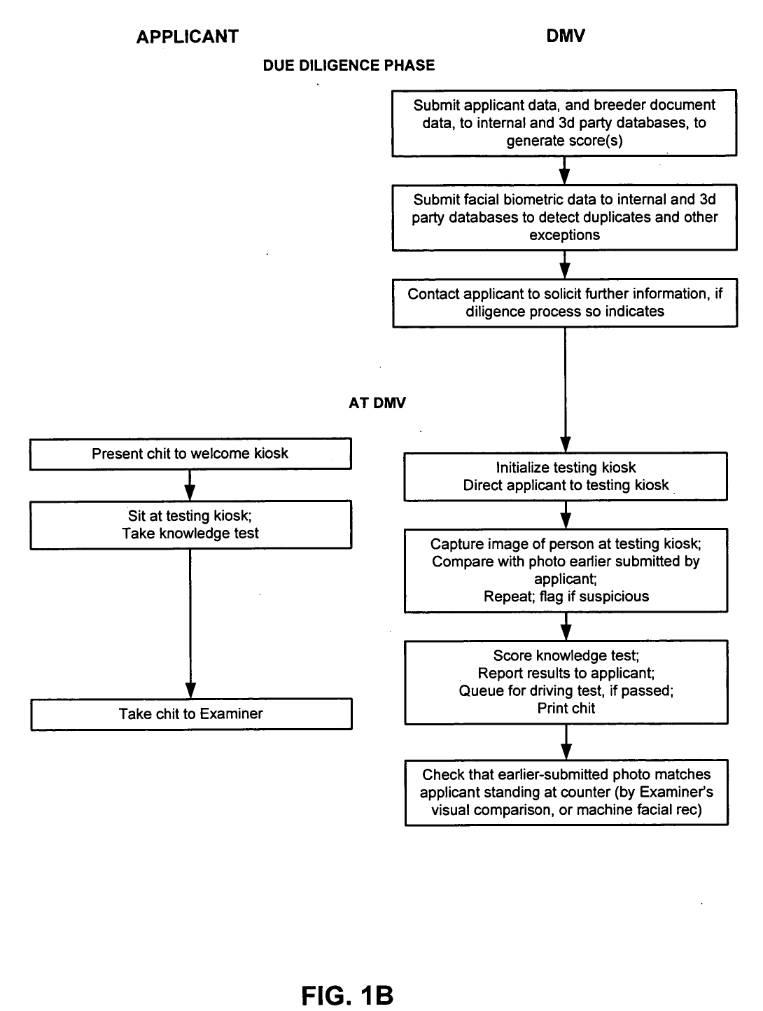

FIGS. 1A and 1B detail an illustrative driver license issuance process, including a “Preparation” phase, a “Remote Application” phase,” a “Due Diligence” phase, and a “At DMV” phase.

In the illustrated “Preparation” phase, an applicant (“Bob”) uses a web browser on a home PC to navigate to a web portal hosted by the state DMV. At the web site Bob can study on-line tutorials and download study guides (e.g., for the knowledge test and the behind-the-wheel test). The web site may also provide information detailing the application process, step-by-step, and can offer practice exams.

In some cases the DMV may want to store Bob's IP address (revealed in the TCP/IP packets received from Bob's computer); however, this is generally not necessary at this preliminary stage.

Sometime later (or immediately), Bob returns to the web portal to actually begin the application process (the “Remote Application” phase). The DMV—through the web site—first inquires as to the precise nature of the transaction. Is it a new driver license or a renewal? Or is it a transfer from another state? Etc. The web site then solicits information from Bob needed for the requested transaction, such as full name, social security number, date and place of birth, address, height, weight, eye color, etc. Bob types this information on his PC, e.g., on a web form. During this process the DMV makes note of the IP address of the computer Bob is using to access the portal site. The information typed by Bob, and the IP address, are stored in one or more database records associated with Bob.

The web site can also request credit card information so that the fee(s) associated with Bob's transaction can be authorized and charged.

As detailed more fully below, Bob can capture his own portrait image, e.g., using his own digital camera, and can submit it to the DMV. Again, the web site can facilitate this process, such as by providing JAVA tools for image quality assurance checking and submission. On receipt, the DMV performs its own Q/A testing, and also computes (and stores) facial parameters from Bob's image. If the submitted image is for some reason unsatisfactory, the web site so-informs Bob, so that he may submit another image. The submitted image is stored in the DMV database. (Any rejected image(s) may be stored in the database as well.)

If Bob has a document scanner, he can scan the “breeder” documents required in his state to establish identity. These may include, e.g., passport, birth certificate, military discharge papers, etc. (more exhaustively detailed in application Ser. No. 10/980,144). Bob can transmit these scans to the DMV (like the portrait photo), together with information he types (e.g., using another web form) characterizing the submitted documents (e.g., document type, date, issuing entity, Bob's name as it appears on the document, etc.). Again, the DMV will check the submitted scan data for compliance with technical standards (e.g., resolution, sharpness, file format, etc.), and will require re-submission if the submitted scans do not pass these tests. If acceptable, the scanned documents are archived in the DMV database. (If Bob doesn't have a scanner, the documents can be scanned when he presents himself at the DMV office.) Generally, the scanned documents are also subjected to an optical character recognition process—extracting the text contents from the scanned document images. Again, this information is stored (and used later, in the Due Diligence phase).

The web portal can then invite Bob to schedule an appointment for his appearance at a particular DMV office. The web site can identify to Bob the DMV office locations nearest his home, or can identify offices near any other location Bob specifies. Once one or more candidate office locations are indicated by Bob as preferable, the web site can present a calendar indicating dates and times at which appointment slots are available. The presented calendar will take into account the DMV resources needed to process Bob's transaction. (If, for example, Bob needs to take a knowledge test, availability of a knowledge testing kiosk is checked. Likewise, if Bob needs to take a behind-the-wheel driving test, the availability of a testing officer is checked.)

If Bob makes an appointment, he can bypass the queued customers who appear at the office unscheduled (the unscheduled applicants may be processed by the DMV as time permits, on a “take a number” basis).

In the illustrated Remote Application phase the web site presents to Bob a chit that he can print on his home printer, and bring with him to his DMV appointment. The chit memorializes—in human readable and/or machine-readable form—some or all of the information known about Bob and his application dossier (including, e.g., particulars about the breeder documents that Bob scanned and submitted electronically).

To conclude the illustrated Remote Application phase, the web site instructs Bob as to next steps in the process. For example, it can tell him that he is to appear at 2:45 p.m. on May 18 at the DMV office on Barbur Blvd., and should bring with him the originals of the birth certificate and passport documents that he scanned and submitted electronically. A map and driving directions to the DMV office may be provided. The instructions may further remind him, e.g., that he is not allowed to answer any cell phone calls while taking the knowledge exam, and that the commute from his home to the DMV office will take about 15 minutes if there is no unusual traffic. The instructions may inform him that, on entering the DMV office, he should present his chit to the Welcome kiosk found just inside the door, to the right. Bob may further be informed that if he fails to check-in at the Welcome kiosk by 3:00, his appointment will be canceled. Finally, he may be informed that if he comes prepared for the 2:45 appoint, he may expect to leave by approximately 3:10 p.m. Bob prints these instructions to bring with him.

FIG. 1B continues with the Due Diligence phase. This aspect of the process is generally transparent to Bob, and involves researching Bob and his purported identity, and checking the breeder documents, to make sure that nothing appears amiss. This process can include checking third party databases, such as credit bureaus, telephone directories, social security databases, etc., to verify that the information submitted by Bob, and the information represented by the breeder documents, is consistent with data maintained by these third parties. (Such a process is more fully detailed in application Ser. No. 10/980,144.) The image submitted by Bob can likewise be checked against an archive of previous driver license images to determine whether a person who looks like Bob has already been issued a driver license. If any ground for suspicion arises, the DMV can contact Bob to solicit further information. If issues are not satisfactorily addressed prior to the appointment, the appointment may be canceled.

The illustrated process concludes with the “At DMV” phase. On entering the DMV office Bob presents his printed chit to the Welcome kiosk. The machine-readable information (e.g., a bar code) on his chit is scanned, and a screen displays a welcoming greeting, and instructions (which may be printed). For example, the Welcome kiosk may initialize a Testing kiosk across the room, and direct Bob to it.

Using the process detailed earlier, the Testing kiosk captures an image of the person appearing before it, and compares it with a photo of Bob (e.g., that submitted in the Remote Application phase). This process may be repeated while Bob interacts with the kiosk to take the knowledge test.

At the conclusion of the knowledge test, the Testing kiosk informs Bob of whether he passed the test. The Testing kiosk also prints a second chit—again having human- and/or machine-readable information printed on it. Assuming Bob passed the knowledge test, the Testing kiosk may immediately inform the testing officer(s) that Bob has passed the knowledge test and is ready for the behind-the-wheel test. Or—if Bob is moving from another state where he was already licensed—a driving test may not be required.

If a driving test is required, at its conclusion the testing officer provides data to the DMV computer indicating Bob's driving score. This may be done, for example, with a wireless PDA carried by the officer and to which the officer identifies himself by using a password, or biometrics, or a cryptographic protocol to assure his identity and authorization.

If Bob has passed all the requisites for issuance of a license, corresponding information is sent to a printing station behind the counter at the DMV office. A printer prints Bob's driver's license—employing the personal information he typed-in on his home computer, and the photo that he took at home. When printed, a clerk picks up the license and calls Bob's name. When Bob presents himself to the counter (perhaps the first time he has dealt with a person in the DMV office) the clerk compares the photo on the just-printed driver license to the person appearing at the counter. The clerk also asks to see the original breeder documents Bob submitted during the Remote Application phase, and visually checks them against the document images presented on the clerk's computer terminal from the scans Bob earlier submitted. If nothing appears amiss, the clerk hands Bob the license.

It will be recognized that driver license issuance processes like that detailed in FIGS. 1A/1B is a great improvement over the prior art. Bob spent a minimum amount of time at the DMV office. The DMV was expecting him when he arrived, and the resources he needed were available when he needed them. The photo on his card is one he likes. Bob is pleased.

The DMV also benefits. Accuracy is increased by permitting Bob to enter some of the information himself—reducing clerical errors. Staffing costs are reduced, since the DMV office needn't be staffed to deal with peak workloads; the workload is moderated by advance scheduling, and staffing can be tailored accordingly. And the need for certain staff functions, such as image capture and breeder document capture, are reduced since these functions are sometimes handled by applicants themselves. Throughput is increased because much of the work is done before applicants appear at the DMV office and, once there, they are quickly advanced through the process.

Finally, and perhaps most importantly, security is enhanced. For example, the advance submission of information by applicants allows the DMV to thoroughly check the authenticity of submitted breeder documents, and applicants' purported identities.

To provide a comprehensive disclosure without unduly lengthening this specification, the above-cited patents and patent applications are incorporated herein by reference.

Having described and illustrated the principles of various novel technologies with reference to specific implementations, it will be recognized that the technologies can be implemented in many other, different, forms and combinations.

For example, while the portrait image printed on the license may be taken at the DMV office (by Bob—using a self-serve photo kiosk, or by DMV personnel), this need not be the case. The applicant may take his or her own photograph, e.g., at home, using his/her own camera, and provide the resulting image—in a standardized image file format (e.g., JPEG, TIFF, etc.)—to the issuing authority. The issuing authority can apply a variety of qualification tests and processes to ensure that the image meets technical specifications and, if the image is technically suitable, accept its use instead of an image taken at the issuing office.

The technical specifications employed to qualify an image can include requirements concerning image size (e.g., pixel dimensions), resolution, focus, lighting, contrast, size of head in frame, placement and orientation of face in frame, absence of background clutter, absence of glare from glasses, presence of open eyes (no blink), absence of sunglasses or concealing head/facial coverings, and suitability for extraction of facial metrics.

A variety of software tools can be employed to check for compliance with such requirements, and to modify certain images to bring them into compliance. Some such tools are commonly available, e.g., image processing software that corrects for poor image contrast. Other tools are specialized to capture of facial portraits for photo ID documents. For example, some such tools find a face within a frame, center the located face, and optionally remove the facial image from the background to obtain a silhouette image. (An example of such a system is shown in 20050031173.) Others analyze the portrait image data to extract facial parameters to assure that such metrics can be satisfactorily determined. Such software tools can be downloaded to the applicant's home computer, and employed there before transmitting an image to the issuing office. Or the tools can be applied at the issuing office, to image data that has not previously been checked by the applicant's computer. Or some testing/processing can be performed at the applicant's computer, and other testing/processing can be performed at the issuing office. The division of testing/processing between the applicant's computer and the issuing office can be complementary, or some tests/processes can be done once at the applicant's computer, and then performed again (e.g., rechecked) at the issuing office.

For routine cases, it is entirely possible for the applicant to have his or her first face-to-face encounter with an employee of the issuing agency at the moment the employee hands the applicant a finished ID card or license—after checking to confirm that the photo on the card matches the applicant to whom it is being handed. (This check can be performed visually, or by imaging the applicant's face at the card pick-up counter, and comparing its facial metrics with the facial metrics derived from the photo on the card.) That is, the personal information may have been entered by the applicant on a terminal—at home or at the agency office. The photo may have been taken in a self-service kiosk at the agency office, or at home. The knowledge test may have been administered by a computer, which relied on biometric verification to confirm the applicant's identity. In some arrangements, and with suitable biometric testing of identity, the knowledge testing may be performed at home.

While certain of the arrangements detailed above were described with reference to particular biometrics, it will be recognized that other biometrics can be used. These include retinal and iris scan data, skin texture data, voiceprint data, fingerprint data, facial parameters, biometrics from other sources, etc. These can be used individually, or in combination. Hashes and other derivatives of such data can also be employed. Capture sensors appropriate to the desired biometrics would of course be used. (Fingerprint sensors are increasingly common as peripherals for home computers; Bob could easily submit his fingerprint—from home—to the DMV.)

While “over-the-counter” issuance arrangements have been particularly considered in the foregoing discussion, the technology detailed herein is likewise applicable with so-called “central issue” systems. In such an arrangement, before the centrally-produced card is mailed to the applicant, certain further security precautions can be undertaken. For example, the address to which the license is to be mailed can be checked to ensure it is not inconsistent with the IP address from which the applicant originally started the process, and/or that it corresponds (through a telephone directory database search) to the telephone number provided by the applicant.

Central issue licenses may require “activation”—much like credit cards—when they are received through the mail by the owner. On receipt, the owner can be required to telephone a toll-free number (e.g., of the DMV) to confirm receipt—using the same telephone number given during the application process. Activation may be required within a certain period of license mailing (e.g., 15 days).

If the license is not activated, then same can be noted in the DMV's database. If the owner later offers the license to a third party, etc. as proof of age or identity (e.g., at an airport, or at a liquor store) and the third party captures machine-readable data from the license and electronically contacts the DMV to verify that the license is valid, the DMV may electronically respond with data indicating that the license is suspect because the owner did not confirm receipt. The third party may then reject the license as insufficient proof of identity or age.

Likewise, if the owner gives the license to a police officer (e.g., at a traffic stop), and the officer learns—through the DMV database—that the license has not been activated, the officer may give the person increased scrutiny.

Instead of telephone activation, a computer-based activation may be employed instead. For example, the licensee may navigate to the DMV's web portal, and enter a receipt code included in the envelope with the license.

Email may be sent to the applicant when his/her license is mailed, informing the applicant of this fact, and instructing the applicant to contact the DMV if the license doesn't arrive within, e.g., 5 business days. In addition, or alternatively, an email can be sent, e.g., 5 days after the physical license is mailed, reporting that the applicant should have received the license already, and if not, to contact the DMV.

The logical linking of first and second data—on a common document or between two different data carriers—can be applied to all of the documents and data carriers contemplated herein.

While reference was made to various forms of machine-readable data (e.g., 2D bar codes, RFIDs, magnetic stripes, digital watermarking, etc.) in the context of vehicle registration documents, it will be recognized that any marking or identification technology can be employed with any the arrangements contemplated herein.

Likewise, while the specification focused on documents as data carriers—with occasional reference to other data carrying media (e.g., vehicle glass, cell phones, digital audio/video players, etc.), it will be recognized that any data carrier may generally be employed in the arrangements detailed above.

While the specification has made frequent reference to departments of motor vehicles, it will be recognized that the ideas expressed above are not limited in their application to DMVs and their needs. The same ideas likewise find applicability with other entities—government or not. (Moreover, it should be recognized that references to DMVs herein should also be understood to encompass others working on behalf of the DMVs, e.g., private contractors, web hosting firms, etc.)

The processes detailed above can be varied in countless ways, such as the reordering and/or omission/addition of steps, etc. (For example, in Bob's on-line application process, he may submit his scheduling preferences before submitting scanned breeder documents; the printing of one or more chits may be omitted; visual checking of the original breeder documents against the earlier-submitted scans can occur immediately after Bob checks in at the Welcome kiosk; the original breeder documents can be rescanned at the DMV office, etc. Likewise, more information can be stored in the DMV database than is particularly indicated above; indeed, all of the information about the applicant, breeder documents, scheduling, failed tests and document/data submissions, and other aspects of the transaction—down to particular test questions presented and answers given—can be archived in the database.)

As will be recognized by the artisan, the methods and systems described above can be implemented using hardware, software or a combination of hardware and software. The methods may be implemented through use of instructions stored in computer readable media (such as electronic, optical or magnetic storage devices).

While the foregoing description has particularly detailed certain combinations, it should be recognized that these are exemplary only. Applicant expressly intends that the teachings herein also be applied in other combinations, and in combination with the teachings of the patent documents cited herein, yielding further novel combinations. Thus, the novel ideas of the embodiments detailed above should not be regarded as applicable only in the context detailed. Rather, it is the applicant's intent that such ideas be employed in each of the contexts where they are applicable (many of which are set forth in the cited patent documents).

In view of the wide variety of embodiments to which the principles and features discussed above can be applied, I claim as my invention all such modifications as may come within the scope and spirit of the following claims and equivalents thereof.

1. In a method of issuing a photo ID card, an improvement comprising obtaining biometric data from a different identity credential, and employing said obtained biometric data in connection with issuance of said photo ID card.

2. The method of claim 1 in which said employing includes storing data related to said biometric data with the photo ID card.

3. The method of claim 1 in which said employing includes verifying the identity of a person presenting said identity credential by reference to said biometric data.

4. A method to automate issuance of a government-issued credential to an applicant, the credential requiring satisfactory completion of a knowledge test by the applicant, the method including—at a knowledge testing station—presenting a credential token including previously-stored biometric information by which the applicant can be verified, capturing biometric information from the applicant, authenticating the applicant by reference to expected correspondence between the captured biometric information and the credential token biometric information, and, upon such authentication, presenting to said applicant the knowledge test.
